(19) United States
(12) Patent Application Publication
(10) Pub. No.: US 2024/0188795 A1
(43) Pub. Date: Jun. 13, 2024

(54) SYSTEMS AND METHODS FOR REDUCING NOISE IN IMAGERY IN A COMPUTER-ASSISTED MEDICAL SYSTEM

(71) Applicant: Intuitive Surgical Operations, Inc., Sunnyvale, CA (US)

(72) Inventors: Changmeng Liu, Sunnyvale, CA (US); Yu-tai Ray Chen, Fremont, CA (US); Adam Begley, Burlingame, CA (US); Dominique D. Brichard, San Jose, CA (US); Andrew J. Hazelton, San Carlos, CA (US)

(21) Appl. No.: 18/286,873

(22) PCT Filed: Apr. 19, 2022
(86) PCT No.: PCT/US2022/025371

§ 371 Date: Oct. 13, 2023

Related U.S. Application Data
(60) Provisional application No. 63/176,815, filed on Apr. 19, 2021.

(51) Int. Cl. A61B 1/00; A61B 1/045; A61B 1/06; H04N 23/60; H04N 25/673; H04N 25/677

(52) U.S. Cl. CPC A61B 1/000095; A61B 1/00114; A61B 1/045; A61B 1/0655; H04N 23/64; H04N 25/673; H04N 25/677

(57) ABSTRACT

Systems and methods for reducing noise in a computer-assisted medical system are described herein. In certain illustrative examples, a system may direct an imaging device to capture one or more pseudo dark frame images of an environment of the imaging device. The system may determine a noise pattern within the set of one or more pseudo dark frame images associated with a coupling of the imaging device and an image processing system. They system may process, based on the noise pattern, additional images captured by the imaging device.

### RELATED APPLICATIONS

The present application claims priority to U.S. Provisional Patent Application No. 63/176,815, filed Apr. 19, 2021, the contents of which is hereby incorporated by reference in its entirety.

### BACKGROUND INFORMATION

A computer-assisted medical system allows a user to control one or more teleoperated medical instruments to perform a medical procedure on a patient. To this end, the computer-assisted medical system captures and displays imagery (e.g., of a surgical space) to the user. Such imagery may be captured by an imaging device coupled to an image processing system of the computer-assisted medical system. However, such imagery may include noise and other such undesired artifacts.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative method includes directing, by a computing device, an imaging device to capture a pseudo dark frame image of an environment of the imaging device; determining, by the computing device and based on the pseudo dark frame image, a noise pattern associated with a coupling of the imaging device and an image processing system; and performing, by the computing device, an operation based on the noise pattern.

An illustrative system includes an imaging device interface; and a processor configured to direct, after an imaging device is coupled to the interface, the imaging device to capture a pseudo dark frame image of an environment of the imaging device; determine, based on the pseudo dark frame image, a noise pattern associated with the system and the imaging device coupled to the interface; and process, based on the noise pattern, additional images captured by the imaging device coupled to the interface.

An illustrative apparatus includes one or more processors; and memory storing executable instructions that, when executed by the one or more processors, cause the apparatus to direct an imaging device to capture a set of one or more pseudo dark frame images of an environment of the imaging device; determine, based on the set of one or more pseudo dark frame images, a noise pattern associated with a coupling of the imaging device to an image processing system; and provide the noise pattern for processing of additional images captured by the imaging device.

An illustrative non-transitory computer-readable medium storing instructions executable by a processor to direct an imaging device to capture a set of one or more pseudo dark frame images of an environment of the imaging device; determine, based on the set of one or more pseudo dark frame images, a noise pattern associated with a coupling of the imaging device to an image processing system; and provide the noise pattern for processing of additional images captured by the imaging device.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIGS. 1-2 depict illustrative configurations including image processing systems according to principles described herein.

FIG. 3 depicts an illustrative processing system according to principles described herein.

FIG. 4 depicts an illustrative configuration including an image processing system according to principles described herein.

FIGS. 5-6 depict illustrative methods according to principles described herein.

FIGS. 7-8 depict illustrative computer-assisted medical systems according to principles described herein.

FIG. 9 depicts an illustrative computing device according to principles described herein.

### DETAILED DESCRIPTION

Systems and methods for reducing noise in imagery in a computer-assisted medical system are described herein. A computer-assisted medical system may include an image processing system configured to be coupled to an imaging device (e.g., an endoscope). A computing device, which may be part of the image processing system or separate from the image processing system, may direct the imaging device to capture one or more pseudo dark frame images of an environment of the imaging device while the image processing system and the imaging device are coupled together. Based on the pseudo dark frame image(s), the computing device may determine a noise pattern associated with a coupling of the imaging device and the image processing system. The computing device may perform an operation based on the noise pattern. For example, the computing device may process additional images captured by the imaging device based on the noise pattern, as described herein, which processing may reduce noise in the additional images.

To illustrate an example, an imaging device (e.g., an endoscope) may be coupled to an image processing system to capture images, such as images within a body of a patient. The imaging device may include one or more image sensors (e.g., charge-coupled device (CCD) sensors, complementary metal oxide semiconductor (CMOS) image sensors, etc.) used to capture images within the body. In some examples, the image sensor(s) may need to fit within a small imaging device so that the imaging device can fit within a small passageway, such as an anatomic passageway of the body. The image sensor(s) may be positioned at a distal portion of the imaging device in order to capture images within the body, and some of the circuitry used to process the captured images may be positioned at a more proximal location (e.g., in an image processing system) given the size constraints of the distal portion of the imaging device. For example, the imaging device may be coupled to the image processing system via a cable and may transmit an analog data image signal along the cable to the image processing system. The image processing system may transmit a clock signal to imaging device for sampling the analog image data signal. The coupling of the analog image data signal and the clock signal along the length of the cable may result in noise added to images output based on the images captured by imaging device. Specifically, the added noise may include a fixed-pattern noise such as a column fixed-pattern noise (CFPN) that has a noise pattern.

While the CFPN may have a noise pattern, the noise pattern of a particular CFPN may depend on various factors, which may include characteristics of the imaging device, image processing system, and/or cable. Thus, the particular noise pattern may be associated with a coupling of imaging device and image processing system and may vary with a change of either component and/or cable.

To determine the noise pattern associated with a coupling of the imaging device and the image processing system, such as the CFPN noise pattern, a user may perform a calibration in which the imaging device captures a dark frame image, which is an image with no light, such as an image captured with a lens of the imaging device covered or closed. The dark frame image may show which pixels and/or columns are outputting signal (and a magnitude of output signal) despite the lack of input, which information may be used to determine the noise pattern of the CFPN. Alternatively or additionally, the dark frame image may include a pre-calibrated dark frame image (e.g., a dark frame image provided by a manufacturer of the imaging device).

However, as mentioned, the noise pattern of a particular CFPN may depend on various factors, which may include characteristics of the imaging device, image processing system, and cable. Consequently, a change to any of the imaging device, image processing system, or cable (e.g., a change to a coupling of the imaging device and the image processing system) may result in a different noise pattern. Thus, if any of these components are changed, the user may have to perform another calibration to identify the different noise pattern. The performance of the calibration may interrupt a workflow (e.g., a medical procedure) to allow for capture of another dark frame image.

To avoid performing calibrations specifically to capture and use dark frame images to determine noise patterns, systems and methods described herein involve dynamically determining noise patterns based on pseudo dark frame images that may be captured in any environment and/or while an imaging device is being initialized. This dynamic determination of noise patterns may allow the imaging device and/or the image processing system of the computer-assisted medical system to be setup and/or changed during a medical procedure without the user having to perform an operation specifically to calibrate for the noise pattern based on a dark frame image. The dynamic determination of noise patterns and use of the noise patterns to reduce noise in images may provide for accurate image output based on arbitrary and dynamic imaging device and image processing system combinations (e.g., arbitrary and dynamic couplings of imaging devices to image processing systems). Illustrative examples of noise reduction described herein may be more accurate and/or flexible than noise reduction that is based on noise patterns determined from calibrations or pre-calibrations that use true dark frame images.

Various illustrative embodiments will now be described in more detail. The disclosed systems and methods may provide one or more of the benefits mentioned above and/or various additional and/or alternative benefits that will be made apparent herein.

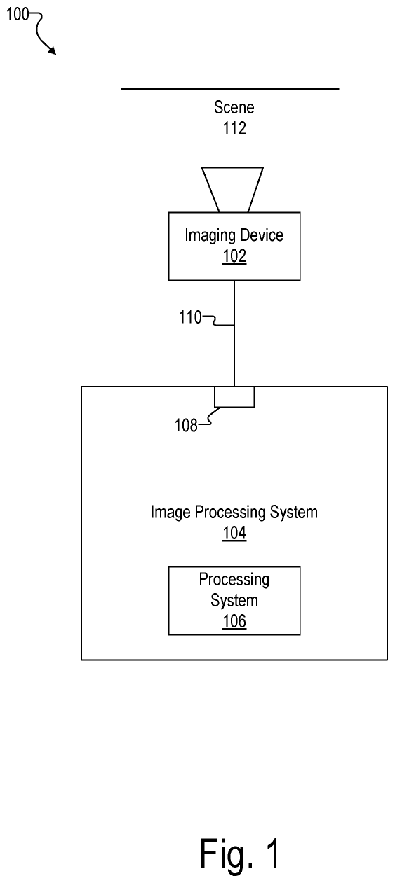

FIG. 1 depicts an illustrative configuration 100 of an imaging device 102 coupled to an image processing system 104 of a computer-assisted medical system. Image processing system 104 may include a processing system 106 and an imaging device interface 108. Imaging device interface 108 may allow image processing system 104 to be coupled to imaging device 102 by a cable 110. Imaging device 102 may be configured to capture images of a scene 112.

Scene 112 may include any environment and/or elements of an environment that may be imaged by imaging device 102. For example, scene 112 may include a tangible real-world scene of physical elements. In certain illustrative examples, scene 112 is associated with a medical procedure such as a surgical procedure. For example, scene 112 may include an environment at a medical site such as a medical facility, operating room, or the like. For instance, scene 112 may include all or part of an operating room in which a medical procedure may be performed on a patient. Additionally or alternatively, scene 112 may include an area on or within a body of a patient, such as an area on which a medical procedure is being performed. For example, imaging device 102 may start imaging a first scene in a first environment at a medical site (e.g., a room in which a medical procedure is performed) and continue imaging a second scene in a second environment within a body of a patient (e.g., a subject of the medical procedure). A medical procedure may include any activity conducted on a patient, such as minimally-invasive surgical procedures, open surgical procedures, non-surgical procedures, diagnostic procedures, therapeutic procedures, procedures in clinical, non-clinical, and/or training settings, etc. A medical procedure may include any activities associated with preparing for, performing, and finalizing the medical procedure, such as pre-procedure activities, intra-procedure activities, and/or post-procedure activities. While such an example is further described herein, one or more principles described herein may be applied to other suitable scenes in other implementations.

Imaging device 102 may include any imaging device configured to capture images (e.g., images constituting frames of a video stream, still images, etc.) of scene 112. For example, imaging device 102 may include an endoscopic imaging device, a video imaging device, an infrared imaging device, a visible light imaging device, a non-visible light imaging device, an intensity imaging device (e.g., color, grayscale, black and white imaging devices), a depth imaging device (e.g., stereoscopic imaging devices, time-of-flight imaging devices, infrared imaging devices, etc.), an ultrasound imaging device, a fluoroscopic imaging device, any other imaging device, or any combination or sub-combination of such imaging devices.

For example, imaging device 102 may include an endoscope configured to capture images within a body of a patient. In some examples, imaging device 102 may be in a lumen of an elongate flexible instrument and removable from the lumen. In some examples, imaging device 102 may be integrated with an elongate flexible instrument. As imaging device 102 captures images within the body, cable 110 may have a sufficient length to keep imaging device 102 connected to image processing system 104 as imaging device 102 traverses areas within the body of the patient (e.g., 0.5 meters, 1 meter, 1.7 meters, or any other suitable length). In some instances, imaging device 102 may be configured to transmit analog signals representative of captured image data along cable 110. The transmission of the analog signals along the length of cable 110 may result in noise added to the captured image data, such as CFPN.

Processing system 106 may be configured to determine a noise pattern using a pseudo dark frame image captured by imaging device 102. The pseudo dark frame image may be an image of an environment of imaging device 102 (e.g., scene 112), as opposed to an image with a lens of imaging device 102 completely covered. Thus, the pseudo dark frame image may be based on at least some input signal to sensors of imaging device 102. For instance, processing system 106 may direct imaging device 102 to capture the pseudo dark frame image by turning off some or all illumination of imaging device 102 (e.g., illumination from light sources of imaging device 102 and/or other light sources communicatively coupled to processing system 106). Additionally or alternatively, processing system 106 may direct imaging device 102 to capture the pseudo dark frame image using a minimal exposure time of imaging device 102. For example, the minimal exposure time may include a minimum exposure time that is capable by imaging device 102 or any suitable substantially short amount of exposure time. By using the minimal exposure time of imaging device 102 with illumination of imaging device 102 turned off, imaging device 102 may capture a pseudo dark frame image of the environment of imaging device 102 that may be used instead of a true dark frame image for determining the noise pattern (e.g., of the CFPN). Determining the noise pattern based on a pseudo dark frame image is further described herein.

Based on the noise pattern, image processing system 104 may be configured to process additional images captured by imaging device 102. For example, image processing system 104 may subtract the noise pattern to correct for the CFPN added by the coupling of imaging device 102 and image processing system 104 via cable 110. As a result, images output by image processing system 104 (e.g., to a user of the computer-assisted medical system that includes image processing system 104) may more accurately depict the additional images as captured by imaging device 102 than conventional systems.

In some examples, the additional images captured by imaging device 102 may be of an additional environment that is different from the environment of the pseudo dark frame image. For instance, after capturing the pseudo dark frame image (or a set of pseudo dark frame images) of a first environment (e.g., a site such as an operating room, etc.), imaging device 102 may be inserted into a body of a patient for a medical procedure. Thus, scene 112 may change from the first environment (e.g., outside the body) to a second environment (e.g., inside the body). Imaging device 102 may capture images of the second environment, which image processing system 104 may process based on the noise pattern. Additionally or alternatively, some or all of the additional images captured by imaging device 102 that are processed based on the noise pattern may be of a same environment as the environment of the pseudo dark frame image. For example, additional images may be captured of the first environment, such as an additional scene of the first environment. Additionally or alternatively, the pseudo dark frame image may be captured in the second environment (e.g., inside the body). For instance, imaging device 102 may be positioned in the body of the patient for the medical procedure when a change is made to a coupling of imaging device 102 and image processing system 104 (e.g., a change in cable 110, image processing system 104, etc.) and/or when imaging device 102 reaches or is near a location at which the medical procedure is to be performed. In such an instance, one or more pseudo dark frame images may be captured at or near a location at which the medical procedure is to be performed and the noise pattern determined based on such pseudo dark frame images.

Image processing system 104 may include any suitable processors configured to process image data representative of images, such as images captured by imaging device 102. Processing system 106 may include any suitable processors configured to perform various operations associated with reducing noise in images, as described herein. Examples of suitable processors, image processing system 104, and processing system 106 are further described herein. While configuration 100 shows processing system 106 as a component of image processing system 104, in some examples, image processing system 104 and processing system 106 may be a same system. In other examples, processing system 106 may be a separate system from image processing system 104.

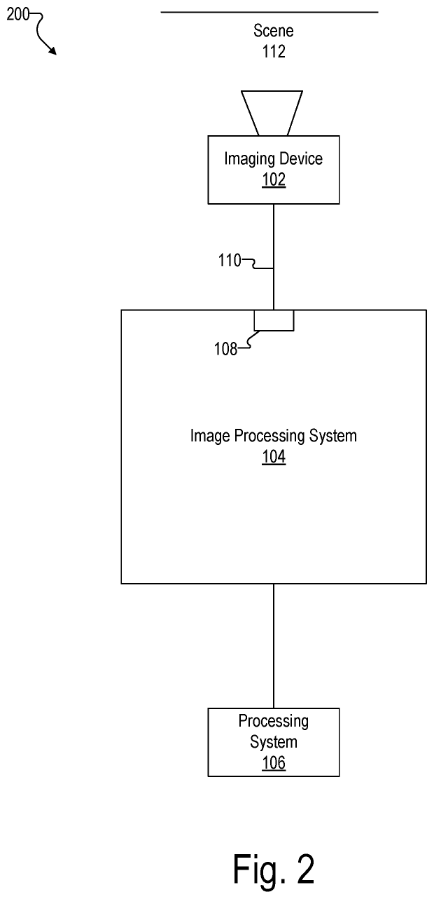

FIG. 2 shows an illustrative configuration 200 that may be similar to configuration 100, including imaging device 102 configured to capture images of scene 112 and coupled to image processing system 104 via cable 110. In configuration 200, however, processing system 106 may be separate from image processing system 104 and communicatively coupled to image processing system 104 and/or imaging device 102.

In such a configuration, processing system 106 may direct imaging device 102 to capture a pseudo dark frame image via image processing system 104 and/or directly if communicatively coupled to imaging device 102. Processing system 106 may be configured to determine a noise pattern based on the pseudo dark frame image and transmit the noise pattern to image processing system 104 for processing additional images captured by image processing system 104. Processing system 106 may transmit the noise pattern to image processing system 104 in any suitable manner.

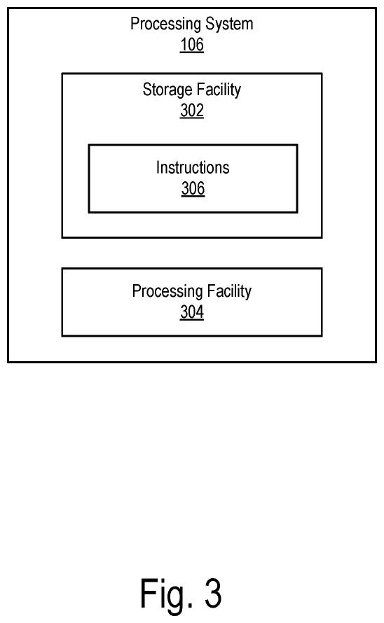

FIG. 3 illustrates an example configuration of processing system 106. Processing system 106 may include, without limitation, a storage facility 302 and a processing facility 304 selectively and communicatively coupled to one another. Facilities 302 and 304 may each include or be implemented by one or more physical computing devices including hardware and/or software components such as processors, memories, storage drives, communication interfaces, instructions stored in memory for execution by the processors, and so forth. Although facilities 302 and 304 are shown to be separate facilities in FIG. 3, facilities 302 and 304 may be combined into fewer facilities, such as into a single facility, or divided into more facilities as may serve a particular implementation. In some examples, each of facilities 302 and 304 may be distributed between multiple devices and/or multiple locations as may serve a particular implementation.

Storage facility 302 may maintain (e.g., store) executable data used by processing facility 304 to perform any of the functionality described herein. For example, storage facility 302 may store instructions 306 that may be executed by processing facility 304 to perform one or more of the operations described herein. Instructions 306 may be implemented by any suitable application, software, code, and/or other executable data instance. Storage facility 302 may also maintain any data received, generated, managed, used, and/or transmitted by processing facility 304.

Processing facility 304 may be configured to perform (e.g., execute instructions 306 stored in storage facility 302 to perform) various operations associated with reducing noise in images for a computer-assisted medical system, such as directing an imaging device to capture a pseudo dark frame image and determining a noise pattern from a pseudo dark frame image.

These and other illustrative operations that may be performed by processing system 106 (e.g., by processing facility 304 of processing system 106) are described herein. In the description that follows, any references to functions performed by processing system 106 may be understood to be performed by processing facility 304 based on instructions 306 stored in storage facility 302.

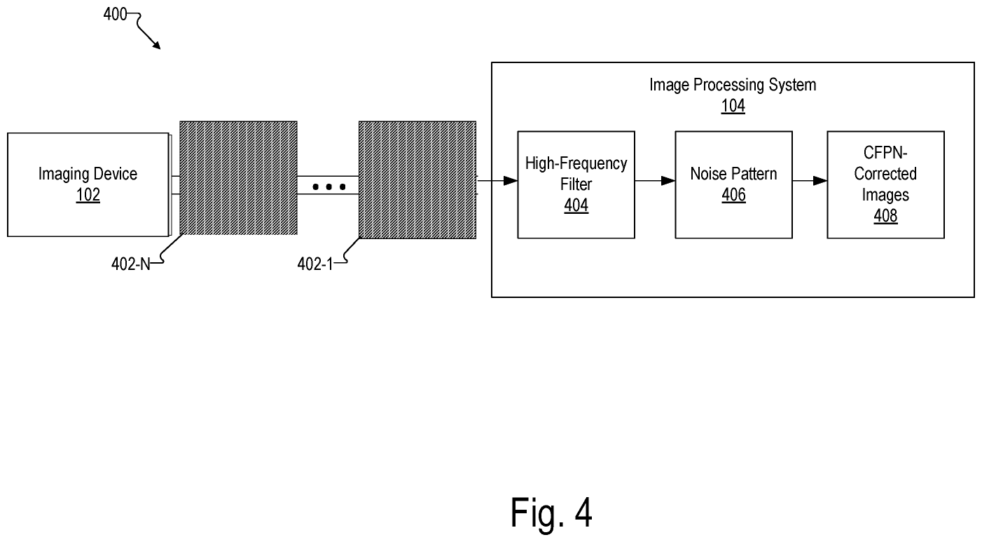

FIG. 4 shows an illustrative configuration 400 of imaging device 102 and image processing system 104. In this example, image processing system 104 includes an implementation of processing system 106 and may perform functions of processing system 106. Image processing system 104 may direct imaging device 102 to capture one or more pseudo dark frame images 402 (e.g., pseudo dark frame image(s) 402-1 through 402-N). In some examples, image processing system 104 may direct imaging device 102 to capture the pseudo dark frame image(s) 402 during an initialization process of imaging device 102. For instance, upon coupling of imaging device 102 to image processing system 104, imaging device 102 may undergo an initialization process including any suitable initialization procedures such as calibrating other parameters of imaging device 102 and initializing sensors of imaging device 102. During the initialization process, image processing system 104 may direct imaging device 102 to capture the one or more pseudo dark frame images 402 for a time period (e.g., approximately three to five seconds). As one example, imaging device 102 may capture a set of 90 pseudo dark frame images 402 over a time period of three seconds. Any other suitable time period may be used, such as a time period for capturing any suitable number of pseudo dark frame images 402. As one example, imaging device 102 may be configured to capture 60 frames per second. In such an example, if a set of 120 pseudo dark frame images 402 are used to determine the noise pattern, image processing system 104 may direct imaging device 102 to capture pseudo dark frame images for 2 seconds.

Imaging device 102 may transmit the set of pseudo dark frame images 402 to image processing system 104. Image processing system 104 may process the set of pseudo dark frame images 402 to determine the noise pattern (e.g., of the CFPN). Image processing system 104 may perform one or more operations based on the noise pattern, such as by removing the noise pattern from one or more additional images captured by the imaging device. The additional images may be captured after the initialization period, with illumination of the imaging device turned on and without using a minimal exposure time.

For instance, image processing system 104 may determine the noise pattern from the pseudo dark frame images 402 by combining the set of pseudo dark frame images 402 in any suitable manner (e.g., determine a mean magnitude of the set of pseudo dark frame images 402) to determine the noise pattern. By using a set of pseudo dark frame images 402, differences between each pseudo dark frame image and a true dark frame image caused by any elements (such as specular lights, reflections of lights, bright objects, etc.) in the environment captured in pseudo dark frame images 402 may be mitigated. Based on the noise pattern, image processing system 104 may perform removing the noise pattern from additional images captured by the imaging device 102 by subtracting the noise pattern from the additional images.

Additionally or alternatively, image processing system 104 may determine the noise pattern from the pseudo dark frame images 402 by applying a high-frequency filter 404 to the set of pseudo dark frame images 402 (and/or a combination of the pseudo dark frame images 402). For example, image processing system 104 may apply a decomposition to each pseudo dark frame image 402 to generate a low-frequency image and a high-frequency component. As the CFPN is carried in the high-frequency component, image processing system 104 may filter the high-frequency component and apply a column noise estimator to the high-frequency component (e.g., using a pyramid representation or any other suitable algorithms) to determine a noise pattern 406 of the CFPN. The characteristic of CFPN carried in high frequency component can be separated to magnitude and phase of the CFPN in each BGGR individual channel. Additionally or alternatively, the magnitude and phase of CFPN of specific frequencies can be detected for every single column. Based on the determined noise pattern, image processing system 104 may perform removing the noise pattern from additional images captured by the imaging device 102. For example, after determination of the phase and amplitude of CFPN for each sub channel or column position, the CFPN correction kernel can use these parameters to remove the CFPN components from an additional image (e.g., including Bayer Image data). For any pixel location, the positive Phase CFPN will be subtracted from RAW data, and negative Phase CFPN will be added to the RAW data. Additionally or alternatively, image processing system 104 may perform removing the noise pattern from additional images at any suitable stage of an image processing pipeline. For example, the noise pattern may be used to determine a noise characteristic of the additional images after demosaicing a chromatic and achromatic noise. Additionally or alternatively, the noise pattern may be used to determine a chromatic/achromatic noise characteristic of the additional images after a color correction matrix and/or a tone mapping and gamma correction. The same method of detection/correction of high frequency component(s) also can be used in detection and correction of other types of noise patterns, such as horizontal fixed-pattern noise (or “row fixed-pattern noise” (RFPN)), radial fixed-noise pattern, and/or any other geometric pattern of fixed noise. Additionally, systems and methods described herein may be applied to detect and correct a fixed pattern of defective pixels (e.g., hot pixels or any other suitable pattern of defective pixels).

Using a high-frequency filter to determine a noise pattern may result in a skewed noise pattern in some instances if the images include any low-frequency illumination changes. Such low-frequency illumination changes may be found in images captured by imaging device 102, as imaging device 102 may include a light or other source of illumination on one side of imaging device 102. Such a light source on one side may generate a smooth (low-frequency) light gradient that might not be correctly filtered using a high-frequency filter, resulting in a skewed noise pattern. However, as the set of pseudo dark frame images 402 may be captured with the illumination of imaging device 102 turned off, light sources found in pseudo dark frame images 402 may likely be from other sources, such as specular light sources. As high-frequency filter 404 may filter out such specular light sources, such skewed noise patterns may be avoided. Thus, by applying high-frequency filter 404 to the set of pseudo dark frame images 402, image processing system 104 may accurately determine noise pattern 406.

In some examples, image processing system 104 may turn off application of high-frequency filter 404 after the set of pseudo dark frame images 402 is received. For instance, image processing system 104 may direct imaging device 102 to capture the set of pseudo dark frame images 402 for a time period during the initialization process of imaging device 102. Image processing system 104 may apply high-frequency filter 404, also during the initialization process, and determine noise pattern 406 based on the set of pseudo dark frame images 402. Once noise pattern 406 is determined, image processing system 104 may stop applying high-frequency filter 404 to additional images. Rather, image processing system 104 may use noise pattern 406 to process the additional images and output CFPN-corrected images 408. Additionally or alternatively, image processing system 104 may further process CFPN-corrected images 408 in any suitable manner.

In this manner, as noise pattern 406 may be determined during the initialization process of imaging device 102, image processing system 104 may be configured to reduce noise generated by any particular noise pattern without interrupting a workflow of a user of imaging device 102. For example, the user would not have to physically cover the lens of the imaging device 102 during the procedure to capture a true dark frame image for noise reduction and then uncover the lens after completion of noise reduction. As another example, the user may encounter issues with a particular first imaging device 102 during a medical procedure. The user may consequently switch to a second imaging device 102, disconnecting the first imaging device 102 from image processing system 104 and connecting the second imaging device 102 to image processing system 104. Such a change in configuration may result in a different, second noise pattern 406. But as image processing system 104 is configured to determine the second noise pattern 406 during initialization of the second imaging device 102, the user may continue the medical procedure upon initialization of the second imaging device 102, without having to perform any separate CFPN-specific calibration of the second imaging device 102.

In some examples, imaging device 102 may include sensors that include a subset of pixels that are configured to receive no input. For instance, imaging device 102 may include columns and/or rows of black line pixels, which may be used to calibrate a black level of imaging device 102. Image processing system 104 may be further configured to additionally or alternatively determine noise pattern 406 based on outputs of the black line pixels. For example, the output of the black line pixels may provide information similar to a true dark frame image. Consequently, image processing system 104 may use the black line pixel output as a dark frame image (or set of dark frame images) for determining noise pattern 406. Additionally or alternatively, image processing system 104 may use the black line pixel output in addition to pseudo dark frame images 402 as additional information for determining noise pattern 406.

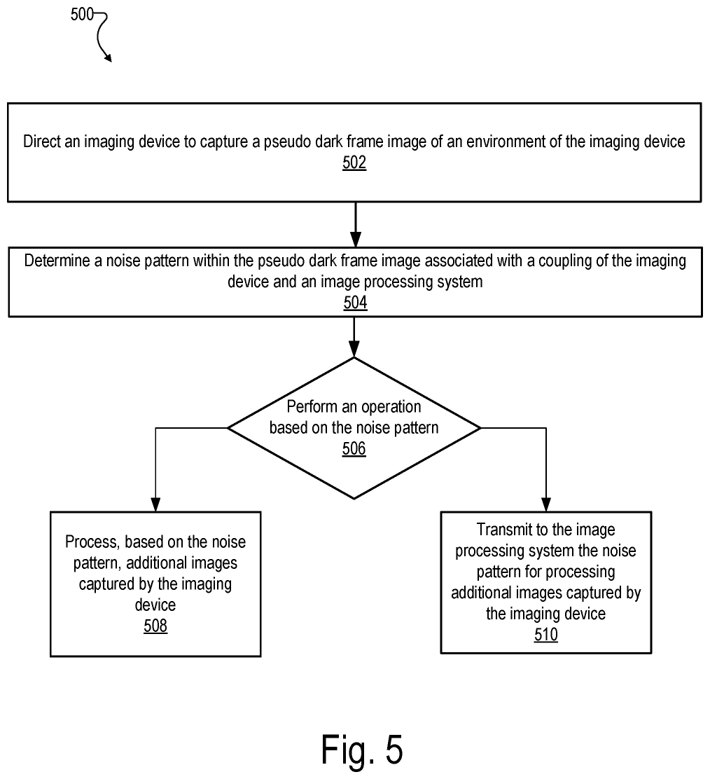

FIG. 5 shows an illustrative method 500 of a computing device of a computer-assisted medical system. While FIG. 5 shows illustrative operations according to one embodiment, other embodiments may omit, add to, reorder, combine, and/or modify any of the operations shown in FIG. 5. One or more of the operations shown in in FIG. 5 may be performed by a computing device such as processing system 106, image processing system 104, any components included therein, and/or any implementation thereof.

In operation 502, a computing device may direct an imaging device to capture one or more pseudo dark frame images of an environment of the imaging device. Operation 502 may be performed in any of the ways described herein.

In operation 504, the computing device may determine a noise pattern within the set of one or more pseudo dark frame images associated with a coupling of the imaging device and an image processing system. Operation 504 may be performed in any of the ways described herein.

In operation 506, the computing device may perform an operation based on the noise pattern. Operation 506 may be performed in any of the ways described herein.

For instance, operation 506 may include operation 508, where the computing device may process, based on the noise pattern, additional images captured by the imaging device. Operation 506 may be performed in any of the ways described herein.

Additionally or alternatively, operation 506 may include operation 510, where the computing device may transmit to the image processing system the noise pattern for processing additional images captured by the imaging device. Operation 510 may be performed in any of the ways described herein.

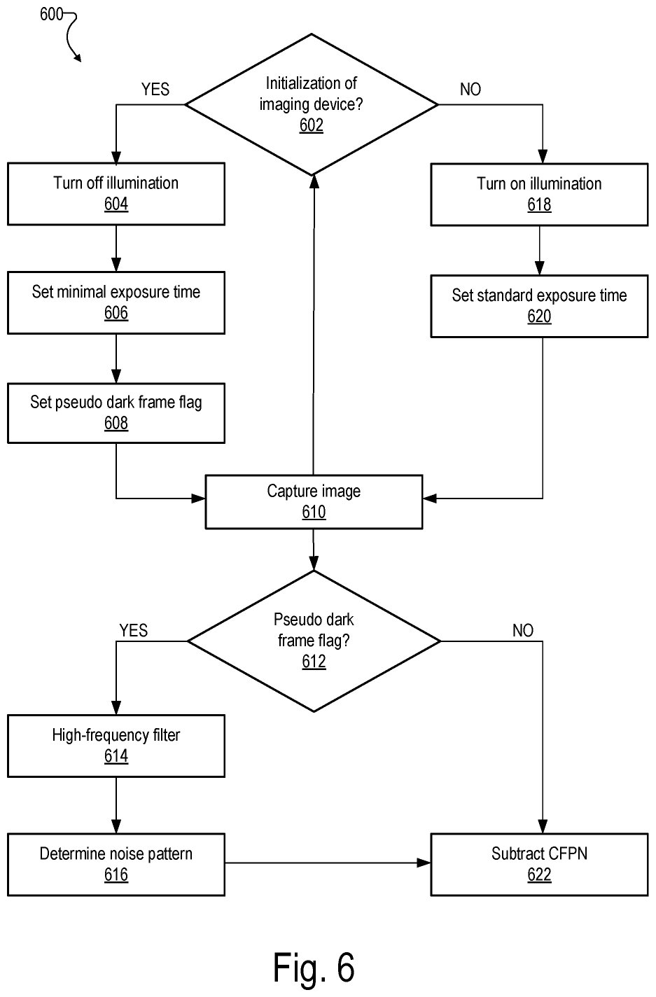

FIG. 6 shows an illustrative method 600 of a computing device of a computer-assisted medical system. While FIG. 6 shows illustrative operations according to one embodiment, other embodiments may omit, add to, reorder, combine, and/or modify any of the operations shown in FIG. 6. One or more of the operations shown in in FIG. 6 may be performed by a computing device such as processing system 106, image processing system 104, any components included therein, and/or any implementation thereof.

In operation 602, a computing device may determine whether an imaging device is being initialized. For example, the imaging device may undergo initialization upon connecting to an image processing system, upon startup of the image processing system, upon a re-initialization request initiated by a user, etc. Operation 602 may be performed in any suitable manner.

If yes, the computing device may perform operation 604. In operation 604, the computing device may direct the imaging device to turn off illumination of the imaging device (e.g., a light emitting diode (LED) or any other light source associated with the imaging device). Operation 604 may be performed in any suitable manner.

In operation 606, the computing device may direct the imaging device to set a minimal exposure time of the imaging device. Operation 606 may be performed in any suitable manner.

In operation 608, the computing device may direct the imaging device to set a flag that may distinguish images as pseudo dark frame images. For instance, the imaging device may be directed to set a flag in metadata of the captured images. Operation 608 may be performed in any suitable manner.

In operation 610, the computing device may direct the imaging device to capture one or more image(s). As the image may be captured with a minimal exposure time and illumination turned off, the captured image(s) may be a pseudo dark frame image(s). As the computing device has directed the imaging device to set a pseudo dark frame flag, the captured pseudo dark frame image(s) may be flagged as such. Operation 610 may be performed in any suitable manner.

In operation 612, the computing device may determine whether received images include the set pseudo dark frame flag. For example, the computing device may examine the metadata of received images for a pseudo dark frame flag setting. Operation 612 may be performed in any suitable manner.

If the pseudo dark frame flag is detected, the computer device may perform operation 614. In operation 614, the computing device may apply a high-frequency filter to the captured pseudo dark frame image. Operation 614 may be performed in any of the ways described herein.

In operation 616, the computing device may determine and/or update a noise pattern based on the filtered pseudo dark frame image. Operation 616 may be performed in any of the ways described herein.

Referring back to operation 602, if the computing device determines that the imaging device is not in an initialization process, the computing device may perform operation 618. In operation 618, the computing device may direct the imaging device to turn on the illumination (e.g., the LED) of the imaging device. Operation 618 may be performed in any suitable manner.

In operation 620, the computing device directs the imaging device to set a standard exposure time, such as an auto exposure or any other suitable exposure setting. Operation 620 may be performed in any suitable manner.

The imaging device may then capture images, as in operation 610, where the captured images do not have the pseudo dark frame flag set. Thus, in operation 612, the computing device may determine that such captured images do not include the set pseudo dark frame flag. Consequently, the computing device may perform operation 622.

In operation 622, the computing device may subtract the noise pattern, which may be accessed (e.g., received, stored, retrieved) from operation 616, from the captured images. As a result, the computing device may reduce noise from the images captured after the imaging device is initialized. Operation 622 may be performed in any suitable manner.

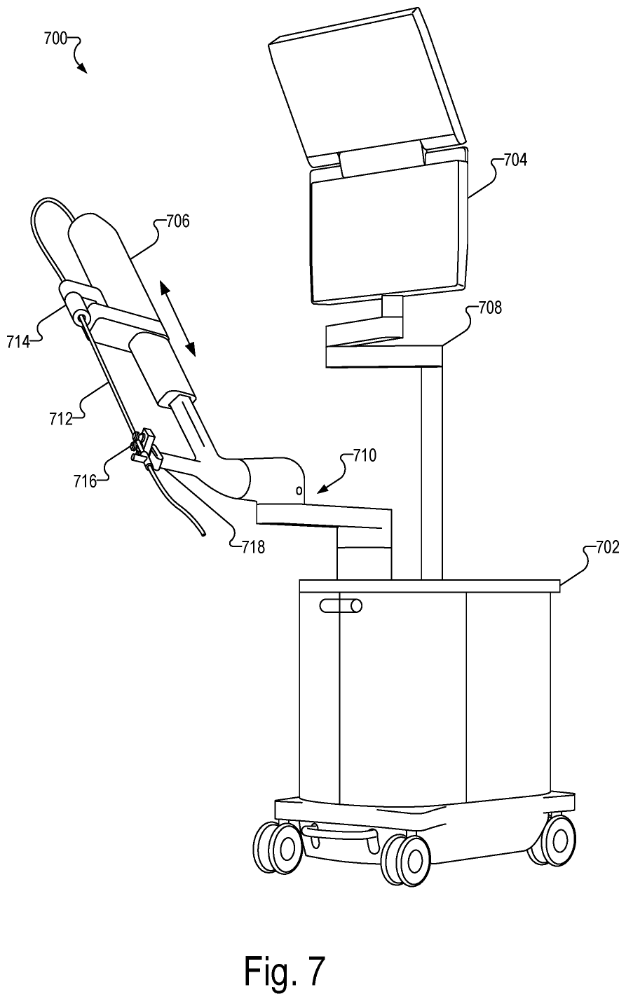

FIG. 7 illustrates an example of a computer-assisted medical system 700 (“medical system 700”) in which image processing system 104 may be implemented. Processing system 106 may be implemented by medical system 700, connected to medical system 700, and/or otherwise used in conjunction with medical system 700. For example, processing system 106 may be implemented by one or more components of medical system 700. As another example, processing system 106 may be implemented by a stand-alone computing system communicatively coupled to medical system 700. Medical system 700 may be utilized by a medical team to perform a computer-assisted medical procedure on a patient.

As shown in FIG. 7, medical system 700 includes a base 702 supporting a display 704 and an instrument manipulator 706. Base 702 may include any structure or assembly suitable for supporting display 704 and instrument manipulator 706. Display 704 may display graphical content to an operator of medical system 700, such as images captured by a vision probe (e.g., an implementation of imaging device 102) inserted into patient anatomy, rendered images of patient anatomy, navigational guidance, etc. Display 704 is attached to base 702 by an arm 708, which may include any structure or assembly for supporting display 704 such that display 704 is viewable by an operator of medical system 700.

Instrument manipulator 706 is attached to base 702 by a setup joint 710. Setup joint 710 may include any structure or assembly that supports instrument manipulator 706 and allows instrument manipulator 706 to be suitably positioned to facilitate insertion and control of an elongate flexible instrument in patient anatomy. To this end, setup joint 710 may include moveable parts, joints, brakes, etc. configured to facilitate suitable positioning of instrument manipulator 706 and an elongate flexible instrument relative to the patient anatomy.

Instrument manipulator 706 may be configured to manipulate an elongate flexible instrument 712, including inserting elongate flexible instrument 712 into patient anatomy. To this end, flexible instrument manipulator 706 may include one or more actuators such as one or more servomotors (not shown) configured to actuate to cause a carriage 714 to which the proximal end of elongate flexible instrument 712 is connected to translate along an insertion axis.

A guide device 716 may be implemented by medical system 700. For example, guide device 716 may be mounted to a mount or docking spar 718 that is attached to setup joint 710. As shown, guide device 716 and docking spar 718 are positioned distal of carriage 714. Guide device 716 and docking spar 718 may be positioned proximate to patient anatomy, and guide device 716 may guide elongated flexible instrument 712 during insertion into the patient anatomy.

Image processing system 104 and/or processing system 106 may be implemented by or communicatively coupled to medical system 700 and may be configured to perform one or more of the operations described herein to direct an imaging device positioned at the distal end of elongate flexible instrument 712 to capture images of an environment of the imaging device and process the captured images in any of the ways described herein. Additionally or alternatively, image processing system 104 and/or processing system 106 may be configured to direct an imaging device inserted through a working channel of elongate flexible instrument 712 to capture images of an environment of the imaging device and process the captured images in any of the ways described herein. As the working channel of elongate flexible instrument 712 may be relatively small (e.g., 2 millimeters), the imaging device may be configured to be small enough to fit within the working channel. Transmitting data via a cable to and from the imaging device and performing image processing on image processing system 104 and/or processing system 106 may allow for such a small configuration of the imaging device.

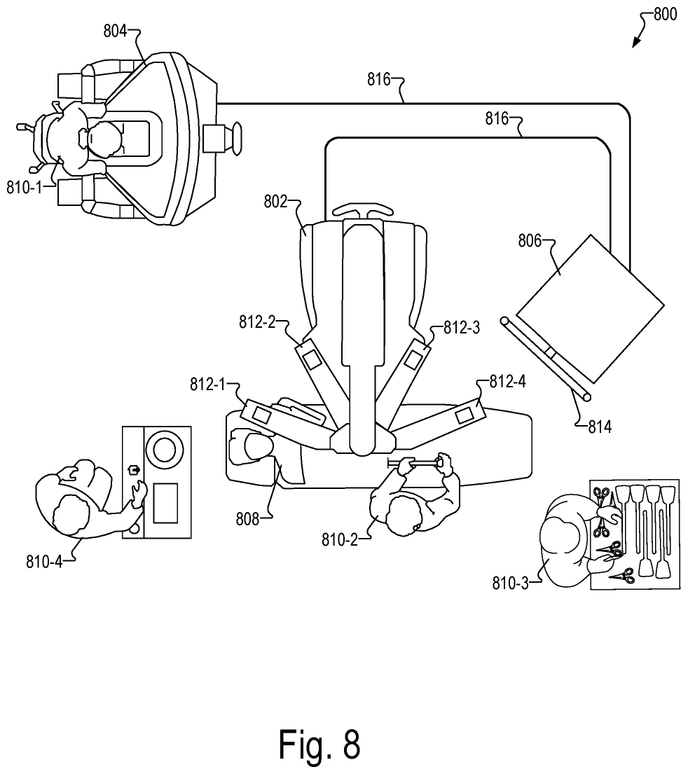

FIG. 8 shows another illustrative computer-assisted medical system 800 (“medical system 800”) in which image processing system 104 may be implemented. Processing system 106 may be implemented by medical system 800, connected to medical system 800, and/or otherwise used in conjunction with medical system 800. For example, processing system 106 may be implemented by one or more components of medical system 800 such as a manipulating system, a user control system, or an auxiliary system. As another example, processing system 106 may be implemented by a stand-alone computing system communicatively coupled to medical system 800.

As shown, medical system 800 may include a manipulating system 802, a user control system 804, and an auxiliary system 806 communicatively coupled one to another. Medical system 800 may be utilized by a medical team to perform a computer-assisted medical procedure on a patient 808. As shown, the medical team may include a surgeon 810-1, an assistant 810-2, a nurse 810-3, and an anesthesiologist 810-4, all of whom may be collectively referred to as “medical team members 810.” Additional or alternative medical team members may be present during a medical session.

While FIG. 8 illustrates an ongoing minimally invasive medical procedure, it will be understood that medical system 800 may similarly be used to perform open medical procedures or other types of surgical procedures that may similarly benefit from the accuracy and convenience of medical system 800. Additionally, it will be understood that the medical session throughout which medical system 800 may be employed may not only include an operative phase of a medical procedure, as is illustrated in FIG. 8, but may also include preoperative (which may include setup of medical system 800), postoperative, and/or other suitable phases of the medical procedure.

As shown in FIG. 8, manipulating system 802 may include a plurality of manipulator arms 812 (e.g., manipulator arms 812-1 through 812-4) to which a plurality of medical instruments may be coupled. Each medical instrument may be implemented by any suitable medical tool (e.g., a tool having tissue-interaction functions), medical tool, imaging device (e.g., an endoscope, an ultrasound tool, etc.), sensing instrument (e.g., a force-sensing medical instrument), diagnostic instrument, or the like that may be used for a computer-assisted medical procedure on patient 808 (e.g., by being at least partially inserted into patient 808 and manipulated to perform a computer-assisted medical procedure on patient 808). While manipulating system 802 is depicted and described herein as including four manipulator arms 812, it will be recognized that manipulating system 802 may include only a single manipulator arm 812 or any other number of manipulator arms as may serve a particular implementation.

Manipulator arms 812 and/or medical instruments attached to manipulator arms 812 may include one or more displacement transducers, orientational sensors, and/or positional sensors used to generate raw (i.e., uncorrected) kinematics information. One or more components of medical system 800 may be configured to use the kinematics information to track (e.g., determine poses of) and/or control the medical instruments, as well as anything connected to the instruments and/or arms.

User control system 804 may be configured to facilitate control by surgeon 810-1 of manipulator arms 812 and medical instruments attached to manipulator arms 812. For example, surgeon 810-1 may interact with user control system 804 to remotely move or manipulate manipulator arms 812 and the medical instruments. To this end, user control system 804 may provide surgeon 810-1 with imagery (e.g., high-definition 3D imagery) of a surgical site associated with patient 808 as captured by an imaging system (e.g., an imaging device such as an endoscope). In certain examples, user control system 804 may include a stereo viewer having two displays where stereoscopic images of a surgical site associated with patient 808 and generated by a stereoscopic imaging system may be viewed by surgeon 810-1. Surgeon 810-1 may utilize the imagery displayed by user control system 804 to perform one or more procedures with one or more medical instruments attached to manipulator arms 812.

To facilitate control of medical instruments, user control system 804 may include a set of master controls. These master controls may be manipulated by surgeon 810-1 to control movement of medical instruments (e.g., by utilizing robotic and/or teleoperation technology). The master controls may be configured to detect a wide variety of hand, wrist, and finger movements by surgeon 810-1. In this manner, surgeon 810-1 may intuitively perform a procedure using one or more medical instruments.

Auxiliary system 806 may include one or more computing devices configured to perform processing operations of medical system 800. In such configurations, the one or more computing devices included in auxiliary system 806 may control and/or coordinate operations performed by various other components (e.g., manipulating system 802 and user control system 804) of medical system 800. For example, a computing device included in user control system 804 may transmit instructions to manipulating system 802 by way of the one or more computing devices included in auxiliary system 806. As another example, auxiliary system 806 may receive and process image data representative of imagery captured by one or more imaging devices attached to manipulating system 802.

In some examples, auxiliary system 806 may be configured to present visual content to medical team members 810 who may not have access to the images provided to surgeon 810-1 at user control system 804. To this end, auxiliary system 806 may include a display monitor 814 configured to display one or more user interfaces, such as images of the surgical site, information associated with patient 808 and/or the medical procedure, and/or any other visual content as may serve a particular implementation. For example, display monitor 814 may display images of the surgical site together with additional content (e.g., graphical content, contextual information, etc.) concurrently displayed with the images. In some embodiments, display monitor 814 is implemented by a touchscreen display with which medical team members 810 may interact (e.g., by way of touch gestures) to provide user input to medical system 800.

Manipulating system 802, user control system 804, and auxiliary system 806 may be communicatively coupled one to another in any suitable manner. For example, as shown in FIG. 8, manipulating system 802, user control system 804, and auxiliary system 806 may be communicatively coupled by way of control lines 816, which may represent any wired or wireless communication link as may serve a particular implementation. To this end, manipulating system 802, user control system 804, and auxiliary system 806 may each include one or more wired or wireless communication interfaces, such as one or more local area network interfaces, Wi-Fi network interfaces, cellular interfaces, etc.

Image processing system 104 and/or processing system 106 may be implemented by or communicatively coupled to medical system 800 and may be configured to perform one or more of the operations described herein to direct an imaging device positioned at a distal end of an instrument coupled to a manipulator arm 812 to capture images of an environment of the imaging device and process the captured images in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Illustrative non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Illustrative volatile storage media include, but are not limited to, RAM (e.g., dynamic RAM).

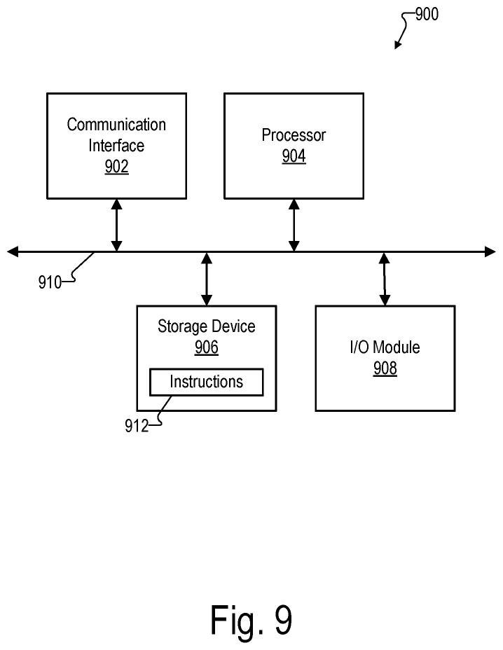

FIG. 9 shows an illustrative computing device 900 that may be specifically configured to perform one or more of the processes described herein. Any of the systems, units, computing devices, and/or other components described herein may implement or be implemented by computing device 900.

As shown in FIG. 9, computing device 900 may include a communication interface 902, a processor 904, a storage device 906, and an input/output (“I/O”) module 908 communicatively connected one to another via a communication infrastructure 910. While an illustrative computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data and/or interpreting, executing, and/or directing execution of one or more of the instructions, processes, and/or operations described herein. Processor 904 may perform operations by executing computer-executable instructions 912 (e.g., an application, software, code, and/or other executable data instance) stored in storage device 906.

Storage device 906 may include one or more data storage media, devices, or configurations and may employ any type, form, and combination of data storage media and/or device. For example, storage device 906 may include, but is not limited to, any combination of the non-volatile media and/or volatile media described herein. Electronic data, including data described herein, may be temporarily and/or permanently stored in storage device 906. For example, data representative of computer-executable instructions 912 configured to direct processor 904 to perform any of the operations described herein may be stored within storage device 906. In some examples, data may be arranged in one or more databases residing within storage device 906.

I/O module 908 may include one or more I/O modules configured to receive user input and provide user output. I/O module 908 may include any hardware, firmware, software, or combination thereof supportive of input and output capabilities. For example, I/O module 908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In some examples, any of the systems, modules, and/or facilities described herein may be implemented by or within one or more components of computing device 900. For example, one or more applications residing within storage device 906 may be configured to direct an implementation of processor 904 to perform one or more operations or functions associated with processing system 106 and/or image processing system 104.

Any of the systems, devices, and/or components thereof may be implemented in any suitable combination or sub-combination. For example, any of the systems, devices, and/or components thereof may be implemented as an apparatus configured to perform one or more of the operations described herein.

In the description herein, various illustrative embodiments have been described. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1-55. (canceled)

56. A method comprising:
directing, by a computing device, an imaging device to capture a pseudo dark frame image of an environment of the imaging device, the directing comprising directing the imaging device to capture an image of the environment of the imaging device using a minimal exposure time of the imaging device;
determining, by the computing device, a noise pattern within the pseudo dark frame image associated with a coupling of the imaging device and an image processing system; and
performing, by the computing device, an operation based on the noise pattern.

57. The method of claim 56, wherein the performing the operation comprises transmitting to the image processing system the noise pattern for processing additional images captured by the imaging device.

58. The method of claim 56, wherein:
the image processing system comprises the computing device; and
the performing the operation comprises processing, based on the noise pattern, additional images captured by the imaging device.

59. The method of claim 58, wherein the additional images captured by the imaging device comprise images captured of an additional environment different from the environment.

60. The method of claim 56, wherein the directing the imaging device to capture the pseudo dark frame image further comprises directing the imaging device to turn off illumination of the imaging device while capturing the pseudo dark frame image.

61. The method of claim 56, further comprising directing, by the computing device, the imaging device to capture a set of pseudo dark frame images of the environment of the imaging device, the set of pseudo dark frame images including the pseudo dark frame image; and
wherein the determining the noise pattern comprises applying a high-frequency filter to the set of pseudo dark frame images.

62. The method of claim 61, wherein the directing the imaging device to capture the set of pseudo dark frame images comprises directing the imaging device to capture the set of pseudo dark frame images for a time period during an initialization process of the imaging device.

63. The method of claim 62, wherein the time period has a duration between approximately three and five seconds.

64. The method of claim 62, wherein the determining the noise pattern comprises determining the noise pattern during the initialization process of the imaging device.

65. The method of claim 56, wherein:
performing the operation comprises removing the noise pattern from an additional image captured by the imaging device;
the pseudo dark image frame is captured with illumination of the imaging device turned off and without covering a lens of the imaging device; and
the additional image is captured with illumination of the imaging device turned on.

66. The method of claim 56, wherein:
the imaging device is in a lumen of an elongate flexible instrument and removable from the lumen; or
the imaging device is integrated with the elongate flexible instrument.

67. The method of claim 56, wherein the noise pattern associated with the coupling of the imaging device and the image processing system is caused by at least one of:
a characteristic of the imaging device;
a characteristic of the image processing system; or
a characteristic of a cable coupling the imaging device to the image processing system.

68. A system comprising:
an imaging device interface; and
a processor configured to:
direct, after an imaging device is coupled to the imaging device interface, the imaging device to capture a pseudo dark frame image of an environment of the imaging device, the directing comprising directing the imaging device to capture an image of the environment of the imaging device using a minimal exposure time of the imaging device;
determine a noise pattern within the pseudo dark frame image associated with the system and the imaging device coupled to the imaging device interface; and
process, based on the noise pattern, additional images captured by the imaging device coupled to the imaging device interface.

69. The system of claim 68, wherein:
the imaging device is coupled to the imaging device interface via a cable; and
the noise pattern is associated with an image signal and a clock signal transmitted on the cable.

70. The system of claim 68, wherein the imaging device is coupled to the imaging device interface via a cable, and the cable is at least 0.5 meters in length.

71. The system of claim 68, wherein:
the pseudo dark frame image of the environment of the imaging device comprises an image of a first scene of the environment; and
the processor is further configured to direct the imaging device to capture the additional images of a second scene of the environment, wherein the second scene is different from the first scene.

72. The system of claim 68, wherein the directing the imaging device to capture the pseudo dark frame image further comprises directing the imaging device to turn off illumination of the imaging device while capturing the pseudo dark frame image.

73. The system of claim 72, wherein the additional images are captured with illumination of the imaging device turned on.

74. A non-transitory computer-readable medium storing instructions executable by a processor to:
direct an imaging device to capture a set of one or more pseudo dark frame images of an environment of the imaging device, the directing comprising directing the imaging device to capture an image of the environment of the imaging device using a minimal exposure time of the imaging device;
determine a noise pattern within the set of one or more pseudo dark frame images associated with a coupling of the imaging device to an image processing system; and
provide the noise pattern for processing of additional images captured by the imaging device.

75. The non-transitory computer-readable medium of claim 74, wherein:
the providing the noise pattern comprises storing the noise pattern in a memory; and
the instructions, when executed, further cause the processor to process, based on the noise pattern, the additional images.
